(19) United States
(12) Patent Application Publication
(10) Pub. No.: US 2020/0001001 A1
(43) Pub. Date: Jan. 2, 2020

(54) LAVAGE DEVICE

(71) Applicant: KAISER TECHNOLOGY CO., LTD., New Taipei City (TW)

(72) Inventor: Mu-Jen WANG, New Taipei City (TW)

(73) Assignee: KAISER TECHNOLOGY CO., LTD., New Taipei City (TW)

(21) Appl. No.: 16/141,271

(22) Filed: Sep. 25, 2018

(30) Foreign Application Priority Data
Jun. 29, 2018 (CN) 201810694519.2

(51) Int. Cl. A61M 3/02

(52) U.S. Cl. CPC A61M 3/0258; A61M 1/0058; A61M 2205/8206; A61M 3/0279

(57) ABSTRACT

A lavage device includes a housing unit, a power module, a transmission unit and an irrigation unit. The housing unit includes a housing body that defines a receiving space having an opening, and a cover that covers openably the opening. The power module is disposed in the receiving space, and is removable from the receiving space via the opening when the cover is opened. The power module includes a motor. The transmission unit includes a driven gear coupled to and driven rotatably by the motor, and a push member driven to reciprocate by the driven gear. The irrigating unit is connected to the transmission unit, and is adapted for drawing liquid through the reciprocate action of the push member and performing irrigation.

### CROSS-REFERENCE TO RELATED APPLICATION

This application claims priority of Chinese Patent Application No. 201810694519.2, filed on Jun. 29, 2018.

### FIELD

The disclosure relates to a lavage device, more particularly to a semi-disposable lavage device used in surgeries.

### BACKGROUND

A conventional lavage device (such as U.S. Pat. Nos. 6,099,494 and 6,059,754) includes a housing, and further includes a plurality of batteries, a motor, a circuit board, a transmission unit, and an irrigation unit, all disposed within the housing. The batteries provide electrical energy and the motor outputs mechanical force, which is transferred through the transmission unit, allowing water to be drawn and dispensed from the irrigation unit.

The conventional lavage device must be disposed of after use. Since the batteries, the motor, the circuit board, the transmission unit, and the irrigation unit are all independently and fixedly installed inside the housing, they are thrown away as well. The disposal of the conventional lavage device does not meet environmental needs and increases medical costs.

### SUMMARY

Therefore, the object of the disclosure is to provide a lavage device that can alleviate at least one of the drawbacks of the prior art.

According to the disclosure, the lavage device includes a housing unit, a power module, a transmission unit, and an irrigation unit.

The housing unit includes a housing body and a cover. The housing body defines a receiving space having an opening. The cover is connected to the housing body and covers openably the opening.

The power module is disposed in the receiving space, and is removable from the receiving space via the opening when the cover is opened. The power module includes an outer shell that is formed with a shaft hole, a motor, a plurality of batteries that are disposed in the outer shell and that are connected electrically to the motor, and a circuit board that is disposed in the outer shell and that is connected electrically to the motor and the batteries. The motor includes a main body disposed in the outer shell, a rotary shaft extending outwardly of the outer shell via the shaft hole, and a coupling member disposed outside of the outer shell and connected to the rotary shaft.

The transmission unit includes a driven gear that is coupled to the coupling member of the motor, and that is driven rotatably by the motor, and a push member that is driven to reciprocate by the driven gear during rotation of the driven gear.

The irrigating unit is connected to the transmission unit, and is adapted for drawing liquid through the reciprocate action of the push member and performing irrigation.

### BRIEF DESCRIPTION OF THE DRAWINGS

Other features and advantages of the disclosure will become apparent in the following detailed description of the embodiment with reference to the accompanying drawings, of which:

FIG. 1 is a perspective view of an embodiment of a lavage device according to the disclosure;

FIG. 2 is a partly exploded perspective view of the embodiment;

FIG. 3 is a perspective view of a power module of the embodiment;

FIG. 4 is a partly cutaway perspective view of the power module of the embodiment;

FIG. 5 is a fragmentary partly exploded perspective view of the power module, a transmission unit and an irrigating unit of the embodiment; and

FIG. 6 is a schematic partly sectional view of the embodiment.

### DETAILED DESCRIPTION

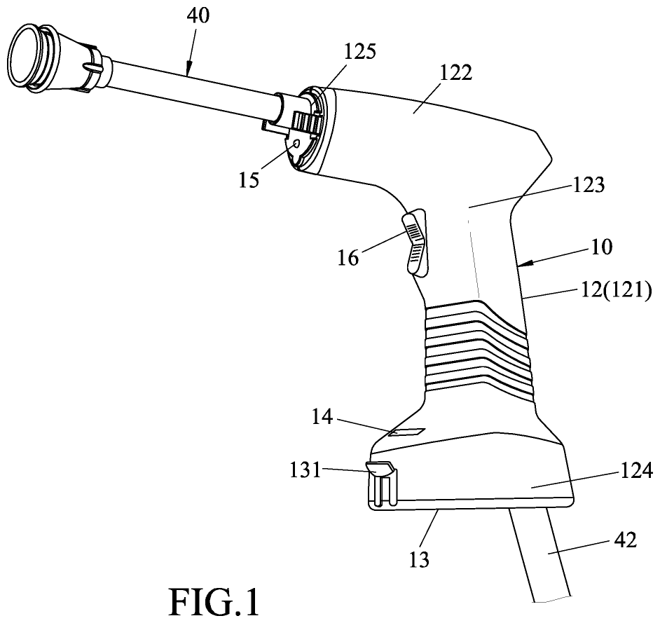

Referring to FIGS. 1, 2, 5, and 6, an embodiment of a lavage device according to the present disclosure includes a housing unit 10, a power module 20, a transmission unit 30 and an irrigation unit 40.

The housing unit 10 includes a housing body 12, a cover 13, a transparent window 14 and a lighting member 15. The housing body 12 defines a receiving space 11 which has an opening 111. The housing body 12 has a grip portion 121, and a barrel portion 122 that is transverse to the grip portion 121. The receiving space 11 extends from the grip portion 121 to the barrel portion 122. The grip portion 121 has a narrow end 123 that is connected to the barrel portion 122, and a widen end 124 that is opposite to the narrow end 123, that has a cross-section larger than that of the narrow end 123, and that is formed with the opening 111. The barrel portion 122 of the housing body 12 has an end that is opposite to the grip portion 121 and that is formed with a through hole 125, the through hole 125 being in spatial communication with the receiving space 11. The cover 13 is connected pivotally to the housing 12, and covers openably the opening 111 of the receiving space 11. The cover 13 has an elastic hook 131 which engages removably with an outer surface of the housing body 12. The transparent window 14 is formed in the widen end 124 of the grip portion 121 of the housing body 12. The lighting member 15 is a light emitting diode (LED), is disposed on the end of the barrel portion 122 of the housing body 12, and is spaced apart from the through hole 125. The housing unit 10 further includes a trigger 16 mounted to the housing body 12, and a contact switch 17 mounted to the housing body 12 and being adjacent to the opening 111.

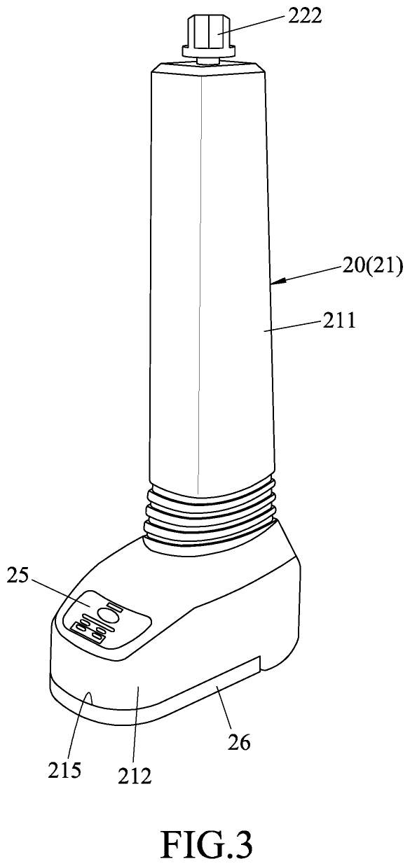

Referring to FIGS. 3, 4, and 6, the power module 20 is disposed in the receiving space 11 and is removable from the receiving space 11 via the opening 111 when the cover 13 is opened. The power module 20 includes an outer shell 21, a motor 22, a plurality of batteries 23 that are disposed in the outer shell 21 and connected electrically to the motor 22, a circuit board 24, a control panel 25, a handle 26 and a plurality of mode switches 27. The outer shell 21 has a shape that corresponds with a shape of the grip portion 121 of the housing body 12, and has a main section 211 and a transverse section 212 that is transverse to the main section 211. The main section 211 has a distal end 214 proximate to the narrow end 123 of the grip portion 121 of the housing 12 and formed with a shaft hole 213. The transverse section 212 is proximate to the opening 111. In this embodiment, the circuit board 24 and the batteries 23 are disposed in the transverse section 212. The motor 22 includes a main body 220 disposed in the distal end 214 of the outer shell 21, a rotary shaft 221 extending outwardly of the outer shell 21 via the shaft hole 213, and a coupling member 222 disposed outside of the outer shell 21 and connected to the rotary shaft 221. The circuit board 24 is disposed in the outer shell 21 and is connected electrically to the motor 22, the batteries 23 and the lighting member 15. The control panel 25 is mounted on the transverse section 212 of the outer shell 21, is registered with the transparent window 14, and displays battery power available in the batteries 23. The handle 26 is connected pivotally to the outer shell 21 and proximate to the opening 111. The transverse section 212 of the outer shell 21 has a retaining groove 215 for retaining the handle 26 therein when the handle 26 is not in use. The plurality of mode switches 27 are mounted on the outer shell 21, are electrically connected to the circuit board 24, and are proximate to the trigger 16.

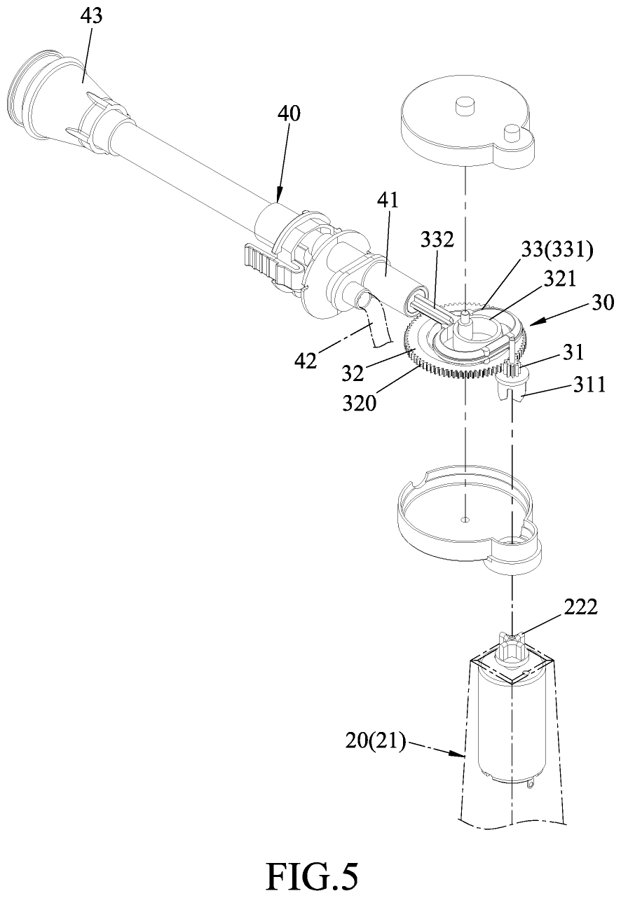

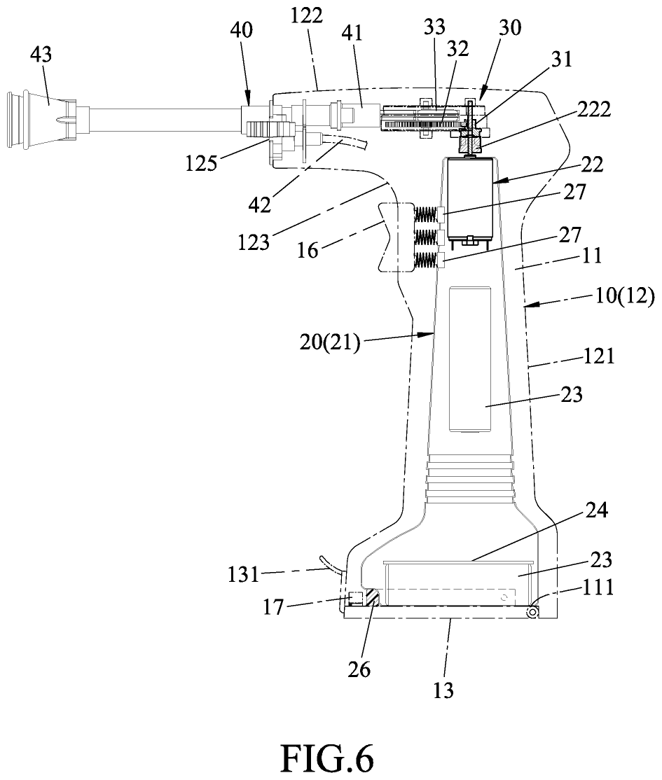

As shown in FIGS. 5 and 6, the transmission unit 30 is connected to the power module 20 and transfer mechanical force. The transmission unit 30 includes a driven gear 31, a transmission gear 32, and a push member 33. The driven gear 31 has a coupling segment 311 that is coupled to the coupling member 222 and driven rotatably by the motor 22. The transmission gear 32 has an annular gear body 320 meshing with the driven gear 31, and a connecting segment 321 connected unconcentrically to the gear body 320. The push member 33 is driven to reciprocate by the driven gear 31 during rotation of the driven gear 31, and has an elliptic guide segment 331 with a substantially oval shape that surrounds the connecting segment 321, and a push rod segment 332 that extends from the guide segment 331. The rotation of the transmission gear 32 drives the push rod segment 332 to reciprocate via the connection between the connecting segment 321 and the guide segment 331.

The irrigation unit 40 is connected to the transmission unit 30, extends through the through hole 125 of the housing body 12, and is adapted for drawing liquid through the reciprocate action of the push member 33 and performing irrigation. The irrigation unit 40 includes a hollow rod-connecting seat 41, an inlet pipe 42 and an outlet pipe 43. The push segment of the transmission unit 30 extends into the rod-connecting seat 41. The inlet pipe 42 and the outlet pipe 43 are connected to the rod-connecting seat 41, such that the reciprocation of the push rod segment 332 of the push member 33 draws liquid to flow from the inlet pipe 42 to the outlet pipe 43.

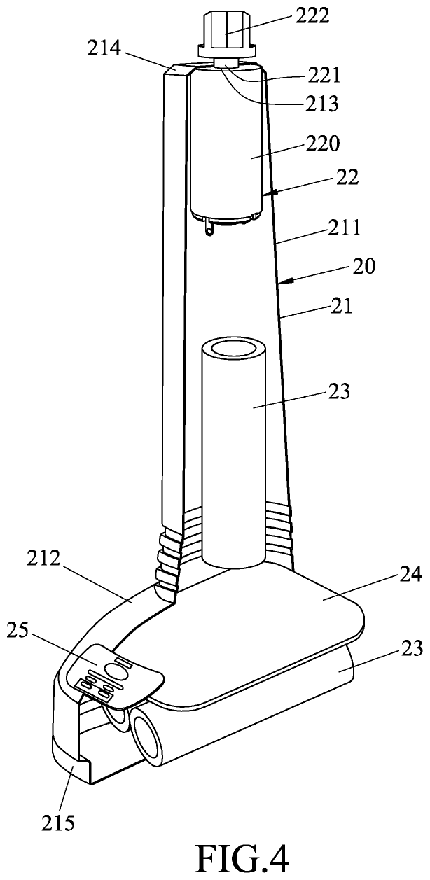

Referring to FIGS. 3 and 4, the power module 20 is substantially modularized. The outer shell 21, the rotary shaft 221 and the coupling member 222 of the motor 22, the control panel 25, and the handle 26 are exposed exteriorly; whereas the main body 220 of the motor 22, the batteries 23 and the circuit board 24 are all hidden within the interior of the outer shell 21.

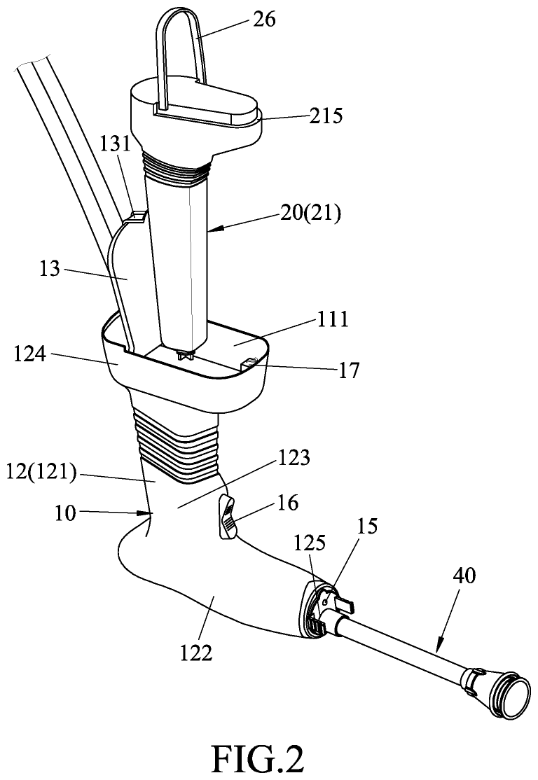

Referring to FIG. 3, battery power available in the batteries 23 may be read from the control panel 25. Further, referring to FIG. 2, by operating the handle 26 pivotally to the outer shell 21, the power module 20 may be conveniently removed from or inserted into the receiving space 11.

When the cover 13 covers the opening 111, the contact switch 17 is triggered to conduct power to the motor 22. The cover 13 cannot cover the opening 111 unless the power module 20 is fully inserted into the housing unit 10. The contact switch 17 being triggered by the cover 13 ensures that the motor 22 cannot be switched on unless the power module 20 has been fully inserted into the housing unit 10, thereby preventing damage of either the power module 20 or the transmission unit 30, and avoiding accidental dispensing of liquid when the trigger 16 is pressed by a user before the power module 20 is fully inserted.

Referring to FIG. 6, after the power module 20 has been inserted into the receiving space 11, the trigger 16 may contact the mode switches 27 to activate the motor 22. Through the connection between the coupling member 222 of the motor 22 and the coupling segment 311 of the driven gear 31, mechanical force from the motor 22 can transfer from the driven gear 31, through the transmission gear 32 to the push member 33, and achieve dispensation of liquid. Furthermore, the trigger 16 is operable to contact a selected one of the mode switches 27 for performing different lavage operations.

Overall, the lavage device according to the present disclosure provides the following benefits:

1. The more costly portion, the power module 20, is produced as a module such that it may be detached after use and reused again, which differs from conventional fully disposable models. This is not only more environmentally friendly, but also reduces costs.

2. The handle 26 allows easier insertion and removal of the power module 20 from the receiving space 11, making the power module 20 easier to reuse. The handle 26 may be retained in the retaining groove 215, stored away when not in use.

3. The lighting member 15 provides a light source to guide direction of lavage operation during use of the lavage device in surgeries.

4. The transverse section 212 of the outer shell 21 uses space effectively in housing the circuit board 24 and the batteries 23.

5. The triggering of the contact switch 17 by the cover 13 closing the opening 111 helps to prevent damage to elements of the power module 20 and the transmission unit 30, as well as prevents accidental dispensing of liquid.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A lavage device comprising:
a housing unit including
a housing body that defines a receiving space having an opening, and
a cover that is connected to said housing body and that covers openably said opening;
a power module disposed in said receiving space, and removable from said receiving space via said opening when said cover is opened, said power module including
an outer shell that is formed with a shaft hole,
a motor that includes a main body disposed in said outer shell, a rotary shaft extending outwardly of said outer shell via said shaft hole, and a coupling member disposed outside of said outer shell and connected to said rotary shaft,
a plurality of batteries that are disposed in said outer shell and that are connected electrically to said motor, and
a circuit board that is disposed in said outer shell and that is connected electrically to said motor and said batteries;
a transmission unit including
a driven gear that is coupled to said coupling member of said motor, and that is driven rotatably by said motor, and
a push member that is driven to reciprocate by said driven gear during rotation of said driven gear; and
an irrigating unit connected to said transmission unit, and adapted for drawing liquid through the reciprocate action of said push member and performing irrigation.

2. The lavage device as claimed in claim 1, wherein:
said housing body has
a grip portion that is formed with said opening, and
a barrel portion that is transverse to said grip portion, and that has an end opposite to said grip portion and formed with a through hole;
said receiving space extends from said grip portion to said barrel portion; and
said irrigating unit extends through said through hole.

3. The lavage device as claimed in claim 2, wherein:
said power module further includes a control panel that is mounted on said outer shell; and
said housing unit further includes a transparent window that is formed in said grip portion of said housing body and that is registered with said control panel.

4. The lavage device as claimed in claim 2, wherein said housing unit further includes a lighting member that is disposed on said end of said barrel portion of said housing body, that is spaced apart from said through hole, and that is connected electrically to said circuit board.

5. The lavage device as claimed in claim 2, wherein:
said grip portion of said housing body has a narrow end that is connected to said barrel portion, and a widen end that is opposite to said narrow end and that has a cross-section larger than that of said narrow end; and
said outer shell of said power module has
a main section that has a distal end proximate to said narrow end of said grip portion of said housing body and formed with said shaft hole, said main body of said motor being disposed in said distal end of said main section of said outer shell, and
a transverse section that is transverse to said main section, said circuit board and at least a portion of said batteries being disposed in said transverse section of said outer shell.

6. The lavage device as claimed in claim 5, wherein:
said power module further includes a handle connected pivotally to said outer shell; and
said transverse section of said outer shell has a retaining groove for retaining said handle therein when said handle is not in use.

7. The lavage device as claimed in claim 1, wherein:
said driven gear has a coupling segment that is coupled to said coupling member of said motor;
said transmission unit further includes a transmission gear that has
an annular gear body meshing with said driven gear, and
a connecting segment connected unconcentrically to said gear body;
said push member has
an elliptic guide segment that surrounds said connecting segment of said transmission gear, and
a push rod segment that extends from said guide segment, such that rotation of said transmission gear drives said push rod segment to reciprocate via the connection between said connecting segment of said transmission gear and said guide segment of said push member;
said irrigating unit includes
a hollow rod-connecting seat, said push rod segment of said push member extending into said rod-connecting seat, and
inlet and outlet pipes that are connected to said rod-connecting seat, such that the reciprocation of said push rod segment of said push member draws liquid to flow from said inlet pipe to said outlet pipe.

8. The lavage device as claimed in claim 1, wherein:
said power module further includes a plurality of mode switches that are mounted on said outer shell, and that are connected electrically to said circuit board; and
said housing unit further includes a trigger that is mounted to said housing body, that is proximate to said mode switches, and that is operable to contact a selected one of said mode switches for performing different lavage operations.

9. The lavage device as claimed in claim 1, wherein said housing unit further includes a contact switch that is mounted to said housing body, that is adjacent to said opening of said receiving space, and that is triggered to conduct power to said motor when said cover covers said opening.
